(19) United States
(12) Patent Application Publication
(10) Pub. No.: US 2019/0352612 A1
(43) Pub. Date: Nov. 21, 2019

(54) ELECTROCHEMICAL BIOSENSOR AND METHOD TO MONITOR BIOLOGICAL CELLS BEHAVIOR IN ACIDIC CONDITIONS

(71) Applicants: Mohammad Abdolahad, Tehran (IR); Alireza Alikhani, Shiraz (IR); Milad Gharooni, Tehran (IR); Hamed Abiri, Mashhad (IR)

(72) Inventors: Mohammad Abdolahad, Tehran (IR); Alireza Alikhani, Shiraz (IR); Milad Gharooni, Tehran (IR); Hamed Abiri, Mashhad (IR)

(73) Assignee: Mohammad Abdolahad, Tehran (IR)

(21) Appl. No.: 16/398,320

(22) Filed: Apr. 30, 2019

Related U.S. Application Data
(60) Provisional application No. 62/686,018, filed on Jun. 17, 2018.

(51) Int. Cl. C12N 5/09; G01N 27/48; C12N 5/071; G01N 33/50

(52) U.S. Cl. CPC C12N 5/0693; G01N 27/48; C12N 2500/60; G01N 33/5044; C12N 5/0602

(57) ABSTRACT

A method for detecting status of biological cells is disclosed. The method includes culturing a plurality of biological cells on a working electrode of an electrochemical biosensor, changing extracellular acidity of the plurality of cultured biological cells by adding an acidic solution onto the working electrode, monitoring an electrochemical response of the plurality of cultured biological cells by monitoring a cyclic voltammetry (CV) diagram from the plurality of cultured biological cells and/or a differential pulse voltammetry (DPV) diagram from the plurality of cultured biological cells, and detecting a status of the plurality of cultured biological cells within one of three status groups including healthy cells, non-metastatic cancer cells, and metastatic cancer cells based on the monitored electrochemical response.

### CROSS-REFERENCE TO RELATED APPLICATION

This application claims the benefit of priority from pending U.S. Provisional Patent Application Ser. No. 62/686,018 filed on Jun. 17, 2018, and entitled “ELECTROCHEMICAL BIOSENSOR TO DISTINGUISH BETWEEN NORMAL AND CANCER CELLS BASED ON MONITORING THEIR ACIDOSIS”, which is incorporated herein by reference in its entirety.

### TECHNICAL FIELD

The present disclosure generally relates to an electrochemical sensor and method for investigation of cell behavior, and particularly, to an electrochemical sensor and method for distinguishing between different types of cells based on their different electrochemical responses due to acidity changes.

### BACKGROUND

Extracellular media containing several interesting analytics may reflect many biological parameters of active bio-agents such as cells and has an important role in modifying their behavior. For example, acidification (reduction in the pH of the extracellular media) may induce many functional changes correlated with the phenotype of the cells, as various studies have shown that acidic tumors are more prone to invasion and metastasis formation. For example, different effects of acidification of the extracellular medium on cell proliferation of healthy and cancerous cells has been investigated by various biological analyzes such as Western Blot analysis.

Electrical-based biosensors have been used as accurate instruments for measuring extracellular parameters of different cell types. Electrical-based biosensors can translate the cell physicochemical phenomena into an electrical signal. Label-free tracking of such physicochemical signatures is of great importance since the presence of the biological markers (like enzyme, antibodies and etc.) in the traditional techniques might seriously affect the resultant response. Moreover, the complexity of chemical modifications would limit the accuracy and reliability of the electrical-based sensor. Whereas, achieving selective label-free interactions between analytics (the complex of the cell and its medium) and the examined interface (the cell culture surface), can lead to great responses with minimized perturbations and maximized precision. Therefore, engineering a bio-electrochemical interface in a label-free bio-sensing manner could be crucial to obtain accurate and stable real-time responses.

Hence, there is a need for a highly accurate sensor and method of use thereof for monitoring a cell's behavior in response to changes in extracellular media, such as acidity changes. Additionally, there is a need for a method for distinguishing between different grades of cells, for example, diagnosing metastatic cells from normal cells by utilizing different effects of a cell's media acidification on a cell's behavior, such as their electrochemical behavior and characteristics.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for detecting status of biological cells. The method may include culturing a plurality of biological cells on a working electrode of an electrochemical biosensor, changing extracellular acidity of the plurality of cultured biological cells by adding an acidic solution onto the working electrode, monitoring an electrochemical response of the plurality of cultured biological cells, and detecting a status of the plurality of cultured biological cells within one of three status groups including healthy cells, non-metastatic cancer cells, and metastatic cancer cells based on the monitored electrochemical response. In an exemplary embodiment, monitoring the electrochemical response of the plurality of cultured biological cells may include monitoring at least one of a cyclic voltammetry (CV) diagram from the plurality of cultured biological cells, a differential pulse voltammetry (DPV) diagram from the plurality of cultured biological cells, and combinations thereof.

In an exemplary implementation, the method may further include fabricating the electrochemical biosensor by forming a nano-roughened surface on the working electrode by roughening surface of the working electrode. In an exemplary embodiment, the nano-roughened surface may include a nano-roughened silicon dioxide (SiO₂) layer including nano-features with diameter of less than 100 nm and height of between 100 nm and 150 nm.

In an exemplary implementation, fabricating the electrochemical biosensor may include growing a silicon dioxide (SiO₂) layer on a silicon substrate, depositing a photoresist layer on the SiO₂ layer, removing the photoresist layer from the SiO₂ layer inside an area associated with the working electrode by patterning the photoresist layer, forming a nano-roughened surface on the SiO₂ layer inside the area associated with the working electrode, removing the photoresist layer from top of the SiO₂ layer, depositing a gold/titanium (Au/Ti) bilayer on the SiO₂ layer, and forming a reference electrode, a counter electrode and the working electrode by patterning the Au/Ti bilayer using photolithography technique. In an exemplary implementation, depositing the gold/titanium (Au/Ti) bilayer on the SiO₂ layer may include depositing a Ti layer on the SiO₂ layer using radio frequency (RF) sputtering system, and depositing an Au layer on the Ti layer using the Radio Frequency (RF) sputtering system.

In an exemplary implementation, forming the nano-roughened surface on the SiO₂ layer inside the area associated with the working electrode may include roughening surface of the area associated with the working electrode by deep reactive ion etching (DRIE) process. Roughening surface of the area associated with the working electrode by DRIE process may include iteratively etching the surface of the area associated with the working electrode and passivating the surface of the area associated with the working electrode. In an exemplary embodiment, etching the surface of the area associated with the working electrode and passivating the surface of the area associated with the working electrode may be iterated subsequently after each other.

In an exemplary implementation, culturing the plurality of biological cells on the working electrode of the electrochemical biosensor may include adhering the plurality of biological cells onto the plurality of nano-features of the nano-roughened surface of the working electrode. In an exemplary implementation, culturing the plurality of biological cells on the working electrode of the electrochemical biosensor may include seeding the plurality of biological cells on the working electrode by adding a cell suspension onto the working electrode, and adhering the cell line to the working electrode by maintaining the electrochemical biosensor with the seeded plurality of biological cells in an incubator. In an exemplary embodiment, the cell suspension may include a cell line in a cell culture medium with normal pH. In an exemplary implementation, culturing the plurality of biological cells on the working electrode of the electrochemical biosensor may further include producing cell lines of a biological tissue by isolating a plurality of cell lines from the biological tissue. In an exemplary implementation, maintaining the electrochemical biosensor in the incubator may include maintaining the electrochemical biosensor with the cell suspension added onto the working electrode in a CO₂ incubator for a time interval between 2 hours and 5 hours.

In an exemplary implementation, changing the extracellular acidity of the plurality of cultured biological cells may include increasing the extracellular acidity of the plurality of cultured biological cells to a pH value in a range between 5.4 and 6.7.

In an exemplary implementation, monitoring the electrochemical response of the plurality of cultured biological cells may include applying a direct current (DC) electrical voltage to the working electrode in a range between −0.8 V and 0.8 V, and extracting the electrochemical response from the electrochemical biosensor. In an exemplary implementation, monitoring the electrochemical response of the plurality of cultured biological cells may include at least one of monitoring a cyclic voltammetry (CV) diagram from the plurality of cultured biological cells, monitoring a differential pulse voltammetry (DPV) diagram from the plurality of cultured biological cells, and combinations thereof.

In an exemplary implementation, monitoring the electrochemical response of the plurality of cultured biological cells may include monitoring a CV diagram from the electrochemical biosensor at least 4 hours after changing the extracellular acidity of the plurality of cultured biological cells, and detecting the status of the plurality of cultured biological cells may include detecting the plurality of cultured biological cells within a group of metastatic cells responsive to lack of an oxidation/reduction peak in the CV diagram. In another exemplary implementation, monitoring the electrochemical response of the plurality of cultured biological cells may include monitoring a CV diagram from the electrochemical biosensor at least 4 hours after changing the extracellular acidity of the plurality of cultured biological cells, and detecting the status of the plurality of cultured biological cells may include detecting the plurality of cultured biological cells within at least one group of healthy cells or non-metastatic cancer cells responsive to observing an oxidation/reduction peak in the CV diagram.

In an exemplary implementation, monitoring the electrochemical response of the plurality of cultured biological cells may include monitoring a set of time-lapsed CV diagrams after changing the extracellular acidity of the plurality of cultured biological cells at time intervals of at least 2 hours, and detecting the status of the plurality of cultured biological cells may include detecting the plurality of cultured biological cells within a group of metastatic cells responsive to increasing a peak current of an oxidation/reduction peak of the CV diagrams in the set of time-lapsed CV diagrams with an increasing rate of less than 3 μA/hr. In another exemplary implementation, monitoring the electrochemical response of the plurality of cultured biological cells may include monitoring a set of time-lapsed CV diagrams after changing the extracellular acidity of the plurality of cultured biological cells at time intervals of at least 2 hours, and detecting the status of the plurality of cultured biological cells may include detecting the plurality of cultured biological cells within the group of healthy cells responsive to an increasing peak current of a set of oxidation/reduction peaks corresponding to the set of time-lapsed CV diagrams with an increasing rate of 7 μA/hr or more. In a further exemplary implementation, monitoring the electrochemical response of the plurality of cultured biological cells may include monitoring a set of time-lapsed CV diagrams after changing the extracellular acidity of the plurality of cultured biological cells at time intervals of at least 2 hours, and detecting the status of the plurality of cultured biological cells may include detecting the plurality of cultured biological cells within the group of non-metastatic cancer cells responsive to an increasing peak current of a set of oxidation/reduction peaks corresponding to the set of time-lapsed CV diagrams with an increasing rate between 3 μA/hr and 7 μA/hr.

In an exemplary implementation, monitoring the electrochemical response of the plurality of cultured biological cells may include monitoring a set of time-lapsed DPV diagrams after changing the extracellular acidity of the plurality of cultured biological cells at time intervals of at least 2 hours, and detecting the status of the plurality of cultured biological cells may include one of detecting the plurality of cultured biological cells within a group of healthy cells responsive to an increasing rate of more than 100% in a set of peak currents of the set of time-lapsed DPV diagrams, detecting the plurality of cultured biological cells within a group of non-metastatic cancer cells responsive to an increasing rate between 50% and 100% in the set of the peak currents of the set of time-lapsed DPV diagrams, and detecting the plurality of cultured biological cells within a group of metastatic cells responsive to an increasing rate of less than 50% in the set of the peak currents of the set of time-lapsed DPV diagrams.

In an exemplary implementation, a method for metastasis diagnosis is disclosed. The method may include fabricating an electrochemical biosensor including a working electrode with a nano-roughened surface, forming a plurality of cultured biological cells on the working electrode by adhering a plurality of biological cells onto the nano-roughened surface of the working electrode, changing extracellular acidity of the plurality of cultured biological cells to a pH value of the extracellular environment of the plurality of cultured biological cells in a range between 5.4 and 6.7 by adding an acidic solution onto the working electrode, applying an electrical voltage to the working electrode in a range between −0.8 V and 0.8 V, extracting a cyclic voltammetry (CV) electrochemical response from the electrochemical biosensor, and detecting a presence of metastatic cells in the plurality of cultured biological cells responsive to a lack of an oxidation/reduction peak in the CV electrochemical response.

In an exemplary implementation, fabricating the electrochemical biosensor including the working electrode with the nano-roughened surface may include forming a nano-roughened surface on an area associated with the working electrode on a silicon dioxide (SiO₂) layer by deep reactive ion etching (DRIE) process, depositing a gold/titanium (Au/Ti) bilayer on the SiO₂ layer, and patterning a reference electrode, a counter electrode and the working electrode on the Au/Ti bilayer using photolithography technique.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates an exemplary method for detecting status of biological cells, consistent with one or more exemplary embodiments of the present disclosure.

FIG. 2 illustrates an exemplary method for fabricating exemplary electrochemical biosensor, consistent with one or more exemplary embodiments of the present disclosure.

FIG. 3A illustrates a schematic view of an exemplary implementation of growing an exemplary SiO₂ layer on an exemplary silicon substrate, consistent with one or more exemplary embodiments of the present disclosure.

FIG. 3B illustrates a schematic view of an exemplary implementation of depositing an exemplary photoresist layer on exemplary SiO₂ layer, consistent with one or more exemplary embodiments of the present disclosure.

FIG. 3C illustrates a schematic view of an exemplary implementation of removing exemplary photoresist layer from top of exemplary SiO₂ layer inside an exemplary area associated with an exemplary working electrode by patterning an exemplary photoresist layer, consistent with one or more exemplary embodiments of the present disclosure.

FIG. 3D illustrates a schematic view of an exemplary implementation of forming an exemplary nano-roughened surface on exemplary SiO₂ layer inside an exemplary area associated with the working electrode, consistent with one or more exemplary embodiments of the present disclosure.

FIG. 3E illustrates a schematic view of an exemplary implementation of removing exemplary photoresist layer from top of exemplary SiO₂ layer, consistent with one or more exemplary embodiments of the present disclosure.

FIG. 3F illustrates a schematic view of an exemplary implementation of depositing an exemplary gold/titanium (Au/Ti) bilayer on exemplary SiO₂ layer, consistent with one or more exemplary embodiments of the present disclosure.

FIG. 3G illustrates a schematic view of an exemplary implementation of forming an exemplary reference electrode, an exemplary counter electrode, and an exemplary working electrode by patterning exemplary Au/Ti bilayer 310, consistent with one or more exemplary embodiments of the present disclosure.

FIG. 4 illustrates an atomic force microscopy (AFM) image of exemplary nano-roughened surface of an exemplary fabricated electrochemical biosensor, consistent with one or more exemplary embodiments of the present disclosure.

FIG. 5A illustrates a field emission scanning electron microscopy (FESEM) image of an exemplary nano-roughened surface of an exemplary working electrode of the fabricated electrochemical biosensor, consistent with one or more exemplary embodiments of the present disclosure.

FIG. 5B illustrates a magnified FESEM image of a small portion of an exemplary nano-roughened surface, consistent with one or more exemplary embodiments of the present disclosure.

FIG. 6 illustrates CV responses of MCF10A cells (top diagrams), MCF7 cells (middle diagrams) and MDA-MB468 cells (bottom diagrams) cultured on nano-roughened working electrode after 8 hours (T=8) and 20 hours (T=20) of incubating in acidic media (pH 6.5 and pH 5.5) compared to normal ambient (pH 7.4), consistent with one or more exemplary embodiments of the present disclosure.

FIGS. 7A-7C illustrate DPV responses of MCF10A cells, MCF7 cells and MDA-MB468 cells, respectively, cultured on nano-roughened working electrode and incubated in different pHs of 7.4, 6.5 and 5.5 for various time intervals of T0 (t=0), T8 (t=8 hours) and T20 (t=20 hours), consistent with one or more exemplary embodiments of the present disclosure.

FIG. 8 illustrates ANPI results for MCF7 cells at pH values of 7.4, 6.5 and 5.5 (left-side images, respectively), and MDA-MB468 cells at pH values of 7.4, 6.5 and 5.5 (right-side images, respectively), consistent with one or more exemplary embodiments of the present disclosure.

FIG. 9 illustrates MMP results for MCF7 cells incubated at pH values of 7.4, 6.5 and 5.5 (top images, respectively), and MDA-MB468 cells incubated at pH values of 7.4, 6.5 and 5.5 (bottom images, respectively), consistent with one or more exemplary embodiments of the present disclosure.

FIG. 10 illustrates nitrite ion (NO₂⁻) release for MCF7 and MDAMB468 cell lines at three different pH values of 7.4 (Control), 6.5 and 5.5, consistent with one or more exemplary embodiments of the present disclosure.

FIG. 11 illustrates ROS release for MCF7 and MDAMB468 cell lines at three different pH values of 7.4 (Control), 6.5 and 5.5, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein, an exemplary bio-electrochemical sensing method is disclosed to monitor the pH-dependent behavior of normal and cancer cells by tracing their biochemical properties, ionic exchanges, and oxidation/reduction interactions. Exemplary method may be utilized to diagnose type of cells, for example, the invasive tumor cells (metastatic cancer cells). The intensity of oxidation/reduction exchanges and electrical current blocking may be the main monitoring parameters of the exemplary method. Exemplary method may utilize a highly accurate label-free electrochemical biosensor including three on-chip integrated electrodes with a working electrode having a nano-roughened surface. Exemplary nano-roughened surface may provide enhanced interactions and adherence between biological cells and the working electrode as well as high resolution electrochemical responses.

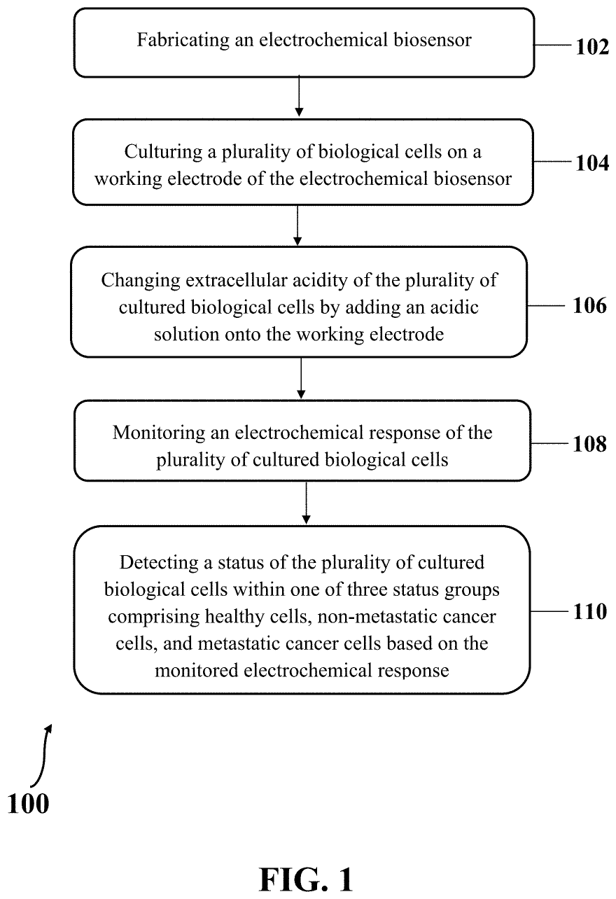

FIG. 1 shows an exemplary method 100 for detecting status of biological cells, consistent with one or more exemplary embodiments of the present disclosure. Exemplary method 100 may include culturing a plurality of biological cells on a working electrode of an electrochemical biosensor (step 104), changing extracellular acidity of the plurality of cultured biological cells by adding an acidic solution onto the working electrode (step 106), monitoring an electrochemical response of the plurality of cultured biological cells (step 108), and detecting a status of the plurality of cultured biological cells within one of three status groups of healthy cells, non-metastatic cancer cells, and metastatic cancer cells based on the monitored electrochemical response (step 110). In an exemplary implementation, method 100 may further include fabricating the electrochemical biosensor (step 102) before culturing the plurality of biological cells on the working electrode of the electrochemical biosensor (step 104) in order to utilize an exemplary electrochemical biosensor having desired features.

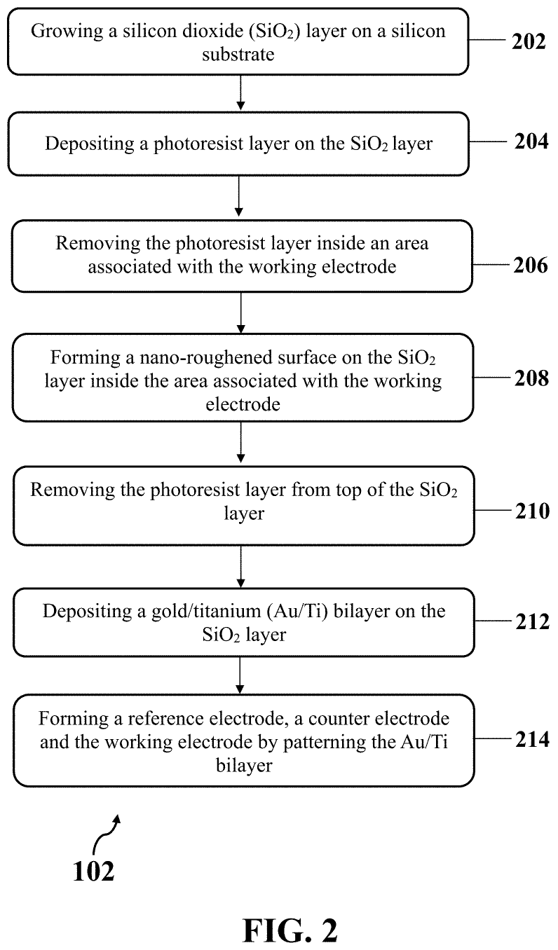

FIG. 2 shows an exemplary method for fabricating exemplary electrochemical biosensor (step 102), consistent with one or more exemplary embodiments of the present disclosure. Specifically, FIG. 2 provides the details for step 102 of FIG. 1. In detail, step 102 may include fabricating an exemplary electrochemical biosensor that may be utilized for detecting status of biological cells based on their electrochemical response to a change in extracellular acidity. In an exemplary implementation, fabricating the electrochemical biosensor may include forming a nano-roughened surface on a working electrode of the electrochemical biosensor by roughening surface of the working electrode. In one implementation, forming the nano-roughened surface may include forming a nano-roughened silicon dioxide (SiO₂) layer on the surface of the working electrode. Forming the nano-roughened surface on the working electrode may include forming a plurality of nano-features (nano-pillars) with diameter of less than about 100 nm and height of between about 100 nm and about 150 nm on the surface of the working electrode.

Referring to FIG. 2, fabricating exemplary electrochemical biosensor may include growing a silicon dioxide (SiO₂) layer on a silicon substrate (step 202), depositing a photoresist layer on the SiO₂ layer (step 204), removing the photoresist layer from top of the SiO₂ layer inside an area associated with the working electrode by patterning the photoresist layer (step 206), forming a nano-roughened surface on the SiO₂ layer inside the area associated with the working electrode (step 208), removing the photoresist layer from top of the SiO₂ layer (step 210), depositing a gold/titanium (Au/Ti) bilayer on the SiO₂ layer (step 212), and forming a reference electrode, a counter electrode and the working electrode by patterning the Au/Ti bilayer (step 214).

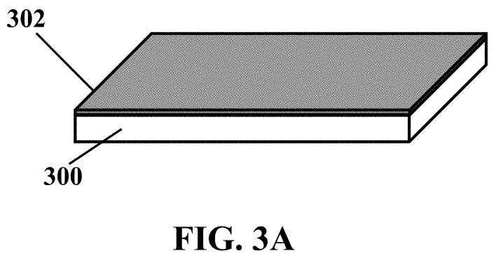

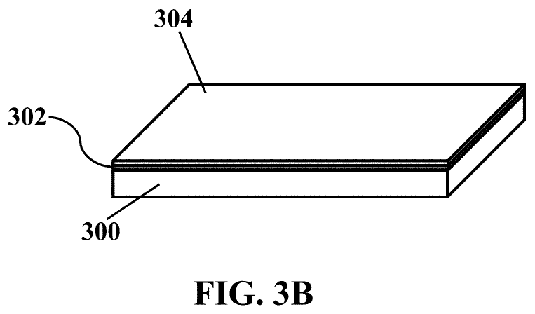

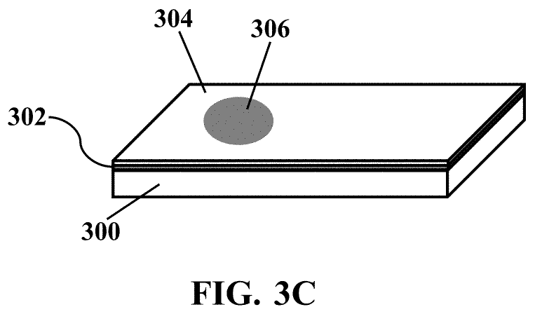

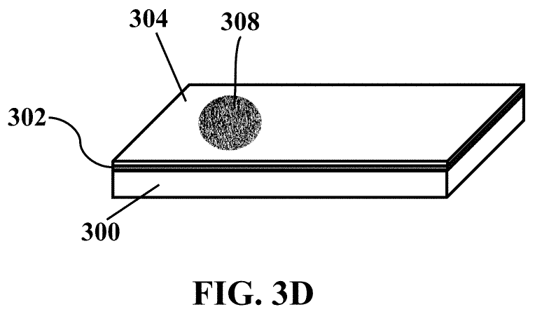

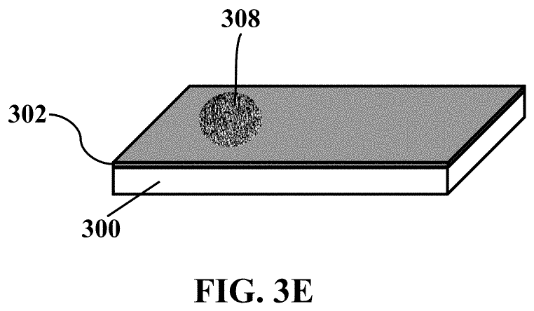

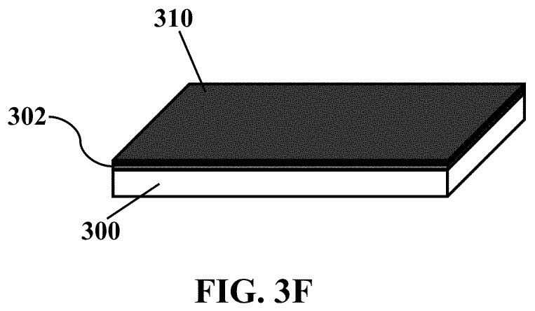

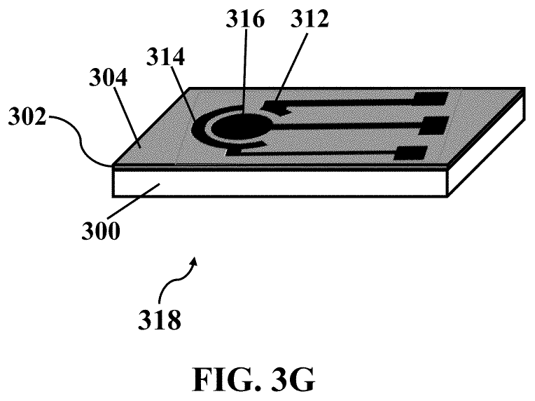

FIGS. 3A-3G show a schematic view of exemplary steps 202 to 214 for fabricating exemplary electrochemical biosensor (step 102), consistent with one or more exemplary embodiments of the present disclosure. FIG. 3A shows a schematic view of an exemplary implementation of growing an exemplary SiO₂ layer 302 on an exemplary silicon substrate 300 (step 202), consistent with one or more exemplary embodiments of the present disclosure. In an exemplary implementation, growing exemplary SiO₂ layer 302 on exemplary silicon substrate 300 may include cleaning silicon substrate 300, and growing SiO₂ layer 302 on silicon substrate 300 using a wet oxidation furnace. Exemplary silicon substrate 300 may be cleaned by a standard solution, for example, a NH₄OH:H₂O₂:H₂O solution with a volume ratio of about 1:1:5. In an exemplary embodiment, SiO₂ layer 302 may include a thin layer of SiO₂ with a thickness of less than about 1 μm. In an exemplar embodiment, SiO₂ layer 302 may include a thin layer of SiO₂ with a thickness of less than about 500 nm. Exemplary SiO₂ layer 302 may be grown on silicon substrate 300 for electrical isolation.

FIG. 3B shows a schematic view of an exemplary implementation of depositing an exemplary photoresist layer 304 on exemplary SiO₂ layer 302 (step 204), consistent with one or more exemplary embodiments of the present disclosure. In an exemplary implementation, photoresist layer 304 with a thickness of about 1 m or less may be deposited on exemplary SiO₂ layer 302.

FIG. 3C shows a schematic view of an exemplary implementation of removing exemplary photoresist layer 304 from top of exemplary SiO₂ layer 302 inside an exemplary area 306 associated with an exemplary working electrode by patterning exemplary photoresist layer 304 (step 206), consistent with one or more exemplary embodiments of the present disclosure. In an exemplary implementation, exemplary area 306 may be designed and considered as an area associated with the working electrode. In an exemplary implementation, exemplary area 306 may be designed in a circular shape by removing a portion of photoresist layer 304 from top of exemplary SiO₂ layer 302 inside exemplary area 306. In an exemplary implementation, exemplary area 306 may be designed in the circular shape in order to allow for a uniform and equal distance between the working electrode and the counter electrode that may provide a uniform electrical current flow between the working electrode and the counter electrode. In addition, the circular shape may provide a maximum area for the working electrode in comparison with other shapes. Moreover, the circular shape may provide a uniform electrical current distribution due to having no geometrical corner or sharp point. In an exemplary implementation, exemplary photoresist layer 304 may be removed inside exemplary area 306 from top of exemplary SiO₂ layer 302 by patterning the photoresist layer 304 using a developer.

FIG. 3D shows a schematic view of an exemplary implementation of forming an exemplary nano-roughened surface 308 on exemplary SiO₂ layer 302 inside exemplary area 306 associated with the working electrode (step 208), consistent with one or more exemplary embodiments of the present disclosure. In an exemplary implementation, forming nano-roughened surface 308 on SiO₂ layer 302 inside exemplary area 306 associated with the working electrode may include roughening surface of exemplary area 306 associated with the working electrode by utilizing a deep reactive ion etching (DRIE) process. A remaining portion of exemplary photoresist layer 304 may act as a mask in a DRIE plasma system that may be utilized for surface nano-roughening process.

In an exemplary implementation, forming nano-roughened surface 308 on SiO₂ layer 302 inside exemplary area 306 associated with the working electrode may include etching the surface of exemplary area 306 associated with the working electrode, and passivating the surface of exemplary area 306 associated with the working electrode. In an exemplary implementation, etching the surface of exemplary area 306 associated with the working electrode and passivating the surface of exemplary area 306 associated with the working electrode may be carried out in an iterative cycle for about twenty iterations.

In an exemplary embodiment, forming nano-roughened surface 308 on SiO₂ layer 302 inside exemplary area 306 associated with the working electrode comprises forming nano-features with diameter of less than 100 nm and height of between 100 nm and 150 nm on SiO₂ layer 302 inside exemplary area 306 associated with the working electrode. In an exemplary embodiment, nano-roughened surface 308 may include a plurality of nano-features (nano-pillars), where each nano-feature (nano-pillar) may have a diameter of less than about 100 nm and a height between about 50 nm and about 200 nm, for example, between about 100 nm and about 150 nm.

In an exemplary implementation, forming nano-roughened surface 308 on SiO₂ layer 302 inside exemplary area 306 associated with the working electrode may include utilizing an anisotropic dry etching method that may include sequential DRIE process. The sequential DRIE process may utilize SF₆ gas for etching the surface of exemplary area 306 associated with the working electrode and a mixture of hydrogen and oxygen with a trace value of SF₆ instead of a polymeric material for passivating the surface of exemplary area 306 associated with the working electrode. This process may benefit from lower plasma power density and higher reactor pressure in comparison with other processes, such as Bosch process. Fluorine ions and radicals may be the primary etching agents to react with silicon atoms from the top surface layer gradually to inside layers, while oxygen and hydrogen ions and radicals combined together may include building blocks to synthesize a complex quasi silicon oxide compound on top of the surface layer. On the other hand, due to the directional nature of plasma, SF₅₊ ions may bombard bottom of the sample and help the removal of bottom passivation layer while sidewalls passivation may be less affected. To achieve a high aspect ratio for nano-pillars, a balance should be maintained between the passivating parameters, especially hydrogen and oxygen gases. Incorporation of H₂/O₂ and SF₆ during the passivating step may lead to formation of a protecting layer over the surface of exemplary area 306 while hydrogen bombardment may help to remove this protective layer from the bottom of the crater.

FIG. 3E shows a schematic view of an exemplary implementation of removing exemplary photoresist layer 304 from top of exemplary SiO₂ layer 302 (step 210), consistent with one or more exemplary embodiments of the present disclosure. In an exemplary implementation, removing exemplary photoresist layer 304 from top of exemplary SiO₂ layer 302 may include peeling exemplary photoresist layer 304 completely; thereby, what remains is exemplary SiO₂ layer 302 with nano-roughened surface 308 on area 306 associated with the working electrode on exemplary silicon substrate 300.

FIG. 3F shows a schematic view of an exemplary implementation of depositing an exemplary gold/titanium (Au/Ti) bilayer 310 on exemplary SiO₂ layer 302 (step 212), consistent with one or more exemplary embodiments of the present disclosure. In an exemplary implementation, depositing exemplary gold/titanium (Au/Ti) bilayer 310 on exemplary SiO₂ layer 302 may include depositing a Ti layer on exemplary SiO₂ layer 302 using radio frequency (RF) sputtering system, and depositing an Au layer on the Ti layer using the Radio Frequency (RF) sputtering system. In an exemplary implementation, depositing exemplary gold/titanium (Au/Ti) bilayer 310 on exemplary SiO₂ layer 302 may include depositing the Ti layer with a thickness of less than 50 nm on exemplary SiO₂ layer 302, and depositing the Au layer with a thickness of less than 500 nm on the Ti layer.

FIG. 3G shows a schematic view of an exemplary implementation of forming an exemplary reference electrode 312, an exemplary counter electrode 314, and an exemplary working electrode 316 by patterning exemplary Au/Ti bilayer 310 (step 214), consistent with one or more exemplary embodiments of the present disclosure. In an exemplary implementation, forming exemplary reference electrode 312, exemplary counter electrode 314, and exemplary working electrode 316 may include patterning exemplary Au/Ti bilayer 310 using a photolithography technique. Therefore, an exemplary electrochemical biosensor 318 may be obtained. As a result, working electrode 316 may be fabricated with a nano-roughened surface that may be coated with an Au layer. Additionally, reference electrode 312 and counter electrode 314 may be coated by Au layer. In an exemplary embodiment, working electrode 316 may have a circular shape and counter electrode 314 may have a ring shape around working electrode 316 in order to increase a resolution of an electrochemical response that may be measured using exemplary electrochemical biosensor 318. Exemplary nano-roughened surface 308 of working electrode 316 may result in a highly efficient interaction and attachment between a sample of biological cells and working electrode 316, and also increasing a resolution of an electrochemical response measured from electrochemical biosensor 318 due to sharp tips of nano-pillars.

Referring again to FIG. 1, step 104 may include culturing a plurality of biological cells on a working electrode of an electrochemical biosensor similar to exemplary working electrode 316 of exemplary fabricated electrochemical biosensor 318 in step 102. In an exemplary implementation, culturing the plurality of biological cells on working electrode 316 of electrochemical biosensor 318 may include adhering the plurality of biological cells onto the plurality of nano-features of exemplary nano-roughened surface 308 of working electrode 316.

In an exemplary implementation, culturing the plurality of biological cells on working electrode 316 of electrochemical biosensor 318 may include seeding the plurality of biological cells on working electrode 316 by adding a cell suspension onto working electrode 316 and adhering/attaching the plurality of biological cells to working electrode 316 by maintaining electrochemical biosensor 318 with the seeded plurality of biological cells in an incubator. In an exemplary embodiment, the cell suspension may include a cell line in a cell culture medium with normal pH of about 7.4. In an exemplary embodiment, the cell line may include at least one of a healthy (normal) cell line, a metastatic cancerous cell line, a non-metastatic cancerous cell line, and combinations thereof.

In an exemplary implementation, seeding the plurality of biological cells on working electrode 316 may include at least one of settling electrochemical biosensor 318 in the cell suspension or by dropping the cell suspension onto electrochemical biosensor 318. In an exemplary implementation, maintaining electrochemical biosensor 318 with the seeded plurality of biological cells in the incubator may include maintaining electrochemical biosensor 318 with the cell suspension added onto working electrode 316 in a CO₂ incubator for a time interval between about 2 hours and about 5 hours. In an exemplary embodiment, the CO₂ incubator may include about 5% CO₂ and about 95% clean air.

In an exemplary implementation, culturing the plurality of biological cells on working electrode 316 of electrochemical biosensor 318 may further include producing cell lines of a biological tissue by isolating a plurality of cell lines from the biological tissue. The produced cell lines may be seeded on working electrode 316; and then, may be adhered onto working electrode 316.

Additionally, step 106 may include changing extracellular acidity of the plurality of cultured biological cells by adding an acidic solution onto the working electrode, for example, working electrode 316. In an exemplary implementation, changing the extracellular acidity of the plurality of cultured cells may include increasing the extracellular acidity of the plurality of cultured cells to a pH value in a range between 5.4 and 6.7 by dropping an acidic solution onto exemplary working electrode 316. In an exemplary implementation, changing the extracellular acidity of the plurality of cultured cells may include lowering pH of a media of the plurality of cultured biological cells by dropping a dilute solution of HCl onto exemplary working electrode 316. In an exemplary embodiment, the diluted solution of HCl may include less than about 0.01 M HCl in less than about 5 ml cell culture media.

In detail, step 108 may include monitoring an electrochemical response of the plurality of cultured biological cells. In an exemplary implementation, monitoring the electrochemical response of the plurality of cultured biological cells may include applying a direct current (DC) electrical voltage to exemplary working electrode 316 in a range between about −0.8 V and about 0.8 V, and extracting the electrochemical response from exemplary electrochemical biosensor 318. In an exemplary implementation, monitoring the electrochemical response of the plurality of cultured biological cells may be carried out using an electrochemical device, for example, an electrochemical workstation or a potentiostat device. The electrochemical device may be connected to exemplary reference electrode 312, counter electrode 314, and working electrode 316 to apply the electrical voltage to exemplary electrochemical biosensor 318 and extract the electrochemical response from electrochemical biosensor 318.

In an exemplary implementation, monitoring the electrochemical response of the plurality of cultured biological cells may include at least one of monitoring a cyclic voltammetry (CV) diagram from the plurality of cultured biological cells, monitoring a differential pulse voltammetry (DPV) diagram from the plurality of cultured biological cells, and combinations thereof.

Additionally, step 110 may include detecting a status of the plurality of cultured biological cells within one of three status groups of healthy cells, non-metastatic cancer cells, and metastatic cancer cells based on the monitored electrochemical response. In an exemplary implementation, detecting the status of the plurality of cultured biological cells may depend on type of the monitored electrochemical response.

In an exemplary implementation, if the monitored electrochemical response includes a CV diagram from the plurality of cultured biological cells, one or more states of the following states may occur. In one implementation, monitoring the electrochemical response of the plurality of cultured biological cells may include monitoring a CV diagram from exemplary electrochemical biosensor 318 at least about 4 hours after changing the extracellular acidity of the plurality of cultured biological cells, and detecting the status of the plurality of cultured biological cells may include detecting the plurality of cultured biological cells within a group of metastatic cells if the CV diagram lacks of an oxidation/reduction peak. In another implementation, monitoring the electrochemical response of the plurality of cultured biological cells may include monitoring a CV diagram from exemplary electrochemical biosensor 318 at least about 4 hours after changing the extracellular acidity of the plurality of cultured biological cells, and detecting the status of the plurality of cultured biological cells may include detecting the plurality of cultured biological cells within at least one group of healthy cells or non-metastatic cancer cells if an oxidation/reduction peak is observed in the CV diagram.

In an exemplary implementation, monitoring the electrochemical response of the plurality of cultured biological cells may include monitoring a set of time-lapsed CV diagrams after changing the extracellular acidity of the plurality of cultured biological cells at time intervals of at least about 2 hours. Accordingly, detecting the status of the plurality of cultured biological cells may include at least one of the following implementations. In one implementation, detecting the status of the plurality of cultured biological cells may include detecting the plurality of cultured biological cells within the group of metastatic cells if an oxidation/reduction peak is observed in more than two CV diagrams of the set of time-lapsed CV diagrams, where a peak current of the oxidation/reduction peaks in the set of time-lapsed CV diagrams may increase with an increasing slope of less than about 3 μA/hr. In another implementation, detecting the status of the plurality of cultured biological cells may include detecting the plurality of cultured biological cells within the group of healthy cells if peak currents of a set of oxidation/reduction peaks corresponding to the set of time-lapsed CV diagrams increases with an increasing slope of about 7 μA/hr or more. In a further implementation, detecting the status of the plurality of cultured biological cells may include detecting the plurality of cultured biological cells within the group of non-metastatic cancer cells if an increasing peak current with an increasing slope between 3 μA/hr and 7 μA/hr is observed in a set of oxidation/reduction peaks corresponding to the set of time-lapsed CV diagrams.

In an exemplary implementation, monitoring the electrochemical response of the plurality of cultured biological cells may include monitoring a set of time-lapsed DPV diagrams after changing the extracellular acidity of the plurality of cultured biological cells at time intervals of at least about 2 hours. Accordingly, detecting the status of the plurality of cultured biological cells may include one of the following cases. In one implementation, detecting the status of the plurality of cultured biological cells may include detecting the plurality of cultured biological cells within the group of healthy cells if an increasing rate of more than about 100% by time increasing is observed for a set of peak currents of the set of time-lapsed DPV diagrams. In another implementation, detecting the status of the plurality of cultured biological cells may include detecting the plurality of cultured biological cells within the group of non-metastatic cancer cells if an increasing rate between about 50% and about 100% by time occurs for the set of the peak currents of the set of time-lapsed DPV diagrams. In a further implementation, detecting the status of the plurality of cultured biological cells may include detecting the plurality of cultured biological cells within the group of metastatic cells if an increasing rate of less than about 50% by time occurs for the set of the peak currents of the set of time-lapsed DPV diagrams occurs.

In an exemplary implementation of the present disclosure, method 100 may be utilized as a method for metastasis diagnosis. The method may include fabricating exemplary electrochemical biosensor 318 that may include exemplary working electrode 316 with exemplary nano-roughened surface 308, forming a plurality of cultured biological cells on working electrode 316 by adhering a plurality of biological cells onto nano-roughened surface 308 of working electrode 316, changing extracellular acidity of the plurality of cultured biological cells to a pH value of the extracellular environment of the plurality of cultured biological cells in a range between about 5.4 and about 6.7 by adding an acidic solution onto working electrode 316, applying an electrical voltage to working electrode 316 in a range between about −0.8 V and about 0.8 V, extracting a cyclic voltammetry (CV) electrochemical response from electrochemical biosensor 318, and detecting a presence of metastatic cells in the plurality of cultured biological cells responsive to a lack of an oxidation/reduction peak in the extracted CV electrochemical response.

In an exemplary implementation, fabricating exemplary electrochemical biosensor 318 may include forming a nano-roughened surface on exemplary area 306 associated with working electrode 316 on exemplary silicon dioxide (SiO₂) layer 302 by a deep reactive ion etching (DRIE) process, depositing exemplary gold/titanium (Au/Ti) bilayer 310 on SiO₂ layer 302, and patterning exemplary reference electrode 312, counter electrode 314 and working electrode 316 on Au/Ti bilayer 310 using photolithography technique.

### Example 1: Fabrication of the Electrochemical Biosensor

In this example, an exemplary electrochemical biosensor similar to electrochemical biosensor 318 was fabricated. A silicon (Si) sample as a substrate was cleaned through RCA1 standard cleaning process using a NH₄OH:H₂O₂:H₂O solution with a volume ratio of 1:1:5, respectively. Subsequently, a thin layer of SiO₂ with a thickness of about 300 nm was grown on the substrate by a wet oxidation furnace. To define a nano-roughened surface on a working electrode of the electrochemical biosensor similar to exemplary working electrode 316, an about 1 micrometer photoresist layer was deposited on the surface of the sample and circle area was patterned by a developer. The anisotropic dry etching method was used for nano-roughening surface of exemplary working electrode 316 that is based on sequential deep reactive ion etching (DRIE) process which utilize SF₆ gas for etching step and a mixture of hydrogen and oxygen with a trace value of SF₆ instead of a polymeric material in passivation step.

Table 1 shows near-optimum conditions for both the passivation and etching sub-cycles in the DRIE process in order to achieve a high aspect ratio for nano-pillars by maintaining a balance between the passivation parameters, especially hydrogen and oxygen gases. The effect of various parameters such as gas flow, plasma power, duration and pressure of each sub-cycle was examined. It was found that a reduction in the flows of both hydrogen and oxygen gases during the passivation step may lead to improper passivation which in turn may lead to an improper formation of a grassy surface. By increasing the passivation time and power, sharp tips were obtained but the total etch rate dropped.

TABLE 1
plasma parameters in nano-roughening process

| Process | Flow (SF₆/O₂/H₂) (sccm) | Power (W) | Time(s) |
|---|---|---|---|
| Etching | 140/0/0 | 130 | 8 |
| Passivation | 13/230/620 | 200 | 50 |

Afterwards, a thin layer of titanium with 20 nm thickness and gold (Au) layer with 200 nm thickness were deposited by RF-sputtering system and patterned by lithography process. Therefore, the working electrode similar to exemplary working electrode 316 was fabricated with nano-roughened surface and reference and counter electrodes similar to reference electrode 312 and counter electrode 314 were coated by Au.

Characterization of the Nano-Roughened Surface:

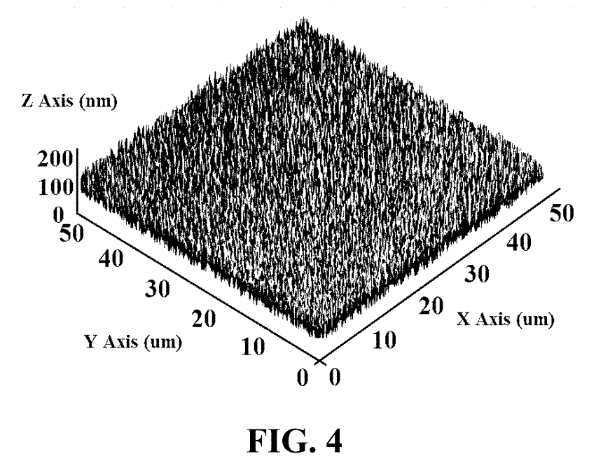

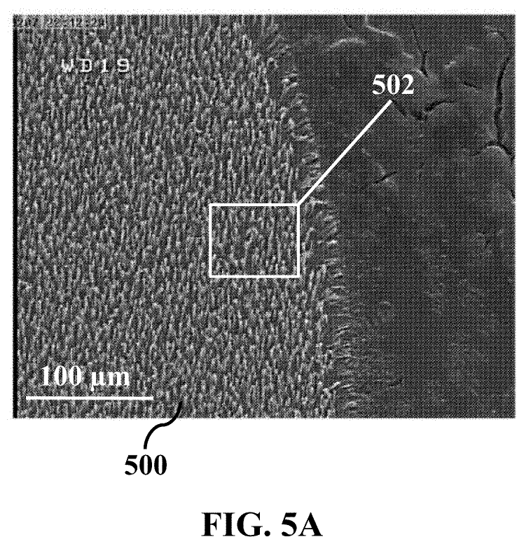

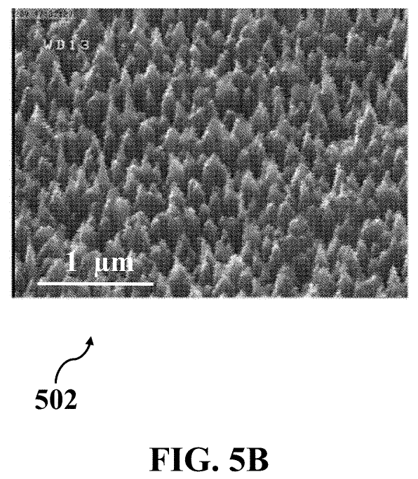

Exemplary fabricated electrochemical biosensor was morphologically characterized by atomic force microscopy (AFM) and field emission scanning electron microscopy (FESEM). FIG. 4 shows an AFM image of exemplary nano-roughened surface of exemplary fabricated electrochemical biosensor, consistent with one or more exemplary embodiments of the present disclosure. FIG. 5A shows a FESEM image of an exemplary nano-roughened surface 500 of exemplary working electrode of the fabricated electrochemical biosensor, consistent with one or more exemplary embodiments of the present disclosure. Moreover, FIG. 5B shows a magnified FESEM image of a small portion 502 of exemplary nano-roughened surface 500 (shown in FIG. 5A), consistent with one or more exemplary embodiments of the present disclosure.

It was observed from FIGS. 4, 5A, and 5B that nano-roughening process induced nano-features with diameter of less than 100 nm and height of about 100 nm to 150 nm. The results revealed that nano-roughening increased the roughness of the surface to about 120 nm that may provide an enhanced interface with the cellular media to better track any ion exchange regarding the biological processes. This high resolution ion exchange tracing could subsequently provide an enhanced sensitivity to detect a minor produced electrical current, which in turn may result in an electrochemical response of the sensor. Additionally, simulation based on analytical software was carried out and demonstrated an electric field enhancement in the biased silicon nano-roughened substrate with respect to a planar smooth silicon surface. The results obtained from finite element simulation showed that the electrical field for nano-roughened surface is about four times greater than that of the planar surface. Hence, the fabrication procedure used herein similar to step 102 of exemplary method 100 hereinabove may provide a quick and simple fabrication process for a high resolution biosensor using electrochemical approach.

### Example 2: Response of the Fabricated Electrochemical Biosensor in the Presence of Different Cells

In this example, electrochemical behavior of different types of biological cells at different acidities of extracellular media were monitored utilizing a method similar to exemplary method 100. An exemplary fabricated electrochemical biosensor that was fabricated according to EXAMPLE 1 was used here.

Cell Culture:

Three types of cell lines, including MCF10A cell line (isolated from normal human breast), MCF7 cell line (isolated from human non-metastatic breast tumor), and MDA-MB468 cell line (isolated from human metastatic breast tumor) were obtained. MCF7 and MDA-MB468 cells were cultured in RPMI-1640 medium supplemented with about 5% fetal bovine serum, and about 1% penicillin/streptomycin. The MCF10A cell line was cultured in DMEM/F12 supplemented with about 10% horse serum, about 1% antibiotic solution, about 0.2% NaHCO₃, insulin (about 5 μg/ml), EGF (about 10 ng/ml) and Hydrocortisone (about 1 g/ml). All cells were maintained in a humidified incubator at about 37° C. containing about 5% CO₂ and the medium was renewed every day. Prior to each electrochemical analysis, medium of cells was removed, then about 1% trypsin was added. After about 5 minutes, RPMI-1640 was added; then, the trypsinized cells were centrifuged for about 5 minute at about 1200 RPM. After removing medium, cells were counted and suspended on working electrode of an exemplary washed electrochemical biosensor, which was settled in a culture dish at normal pH (pH=about 7.4). To minimize the effect of trypsinization, the procedure was taken less than about 4 minutes at room temperature about 20° C. to about 22° C. The electrochemical biosensor was held in an incubator for about 4 hours to achieve cell attachment on working electrode of exemplary electrochemical biosensor.

Cyclic Voltammetry (CV) and Differential Pulse Voltammetry (DPV) Measurement Procedure:

For CV and DPV characterization, three-electrode electrochemical cyclic voltammetry was performed using the electrochemical workstation. Instead of the system electrodes, integrated electrodes of exemplary fabricated electrochemical biosensor were used. CV and DPV were performed between exemplary integrated nano-roughened working electrode 316 and counter electrode 314, with exemplary on-chip reference electrode 312. The reference electrode was calibrated before by Ag/AgCl reference electrode in about 1 mM ferrocene carboxylic acid with about 1 mM potassium chloride solution. CV measurements were performed using DC voltage and no AC frequency was applied. For CV data recording, measurements were carried out at about −0.4 V to about 0.8 V at a scan rate of 100 mv/s. Following addition of two acidic media with pH values of about 5.5 and about 6.5 to the adhered cells on the electrochemical biosensor, CV and DPV signals were extracted at time intervals of about 8 hours and 20 hours after changing acidity. DPV was performed as an alternative approach for CV by removing the common mode (baseline current) in the CV signals in order to observe the complete alterations of current. Since ionic culture media RPMI-1640 (enriched with about 10% FBS) was employed for the culture of cancer cells, the base electrochemical response of this media was considered in all of the analyses by considering an electrochemical response of RPMI-1640 media at about 200 mV.

CV Responses Monitoring and Analysis:

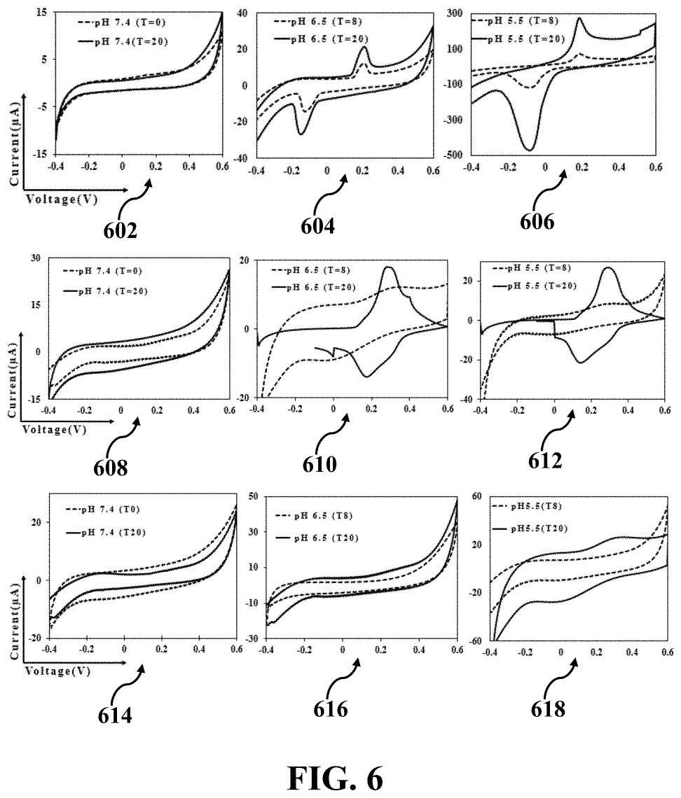

FIG. 6 shows CV responses of MCF10A cells (top diagrams), MCF7 cells (middle diagrams), and MDA-MB468 cells (bottom diagrams) cultured on nano-roughened working electrode after 8 hours (T=8) and 20 hours (T=20) of incubating in acidic media (pH 6.5 and pH 5.5) compared to normal ambient (pH 7.4), consistent with one or more exemplary embodiments of the present disclosure. Curves of diagram 602 show CV signals of MCF10 cells in normal pH. Curves of diagram 604 show semi-reversible anodic/cathodic peaks for MCF10 cells in pH 6.5. Curves of diagram 606 show sharper peaks in MCF10 cells in pH 5.5 which may mean that an ionic electrochemical reaction proceeded in the acidic media and got progressed in the lower pH. Curves of diagram 608 show CV signals for the MCF7 cells in the normal pH. Curves of diagrams 610 and 612 show recorded current measurements for MCF7 cells in pH 6.5 and pH 5.5, respectively that show a detectable trace of peaks at T=20 hours. Curves of diagram 614 show CV signals for the MDA-MB468 cells in the normal pH and curves of diagrams 616 and 618 show recorded measurements for MDA-MB468 cell line in pH 6.5 and pH 5.5, respectively.

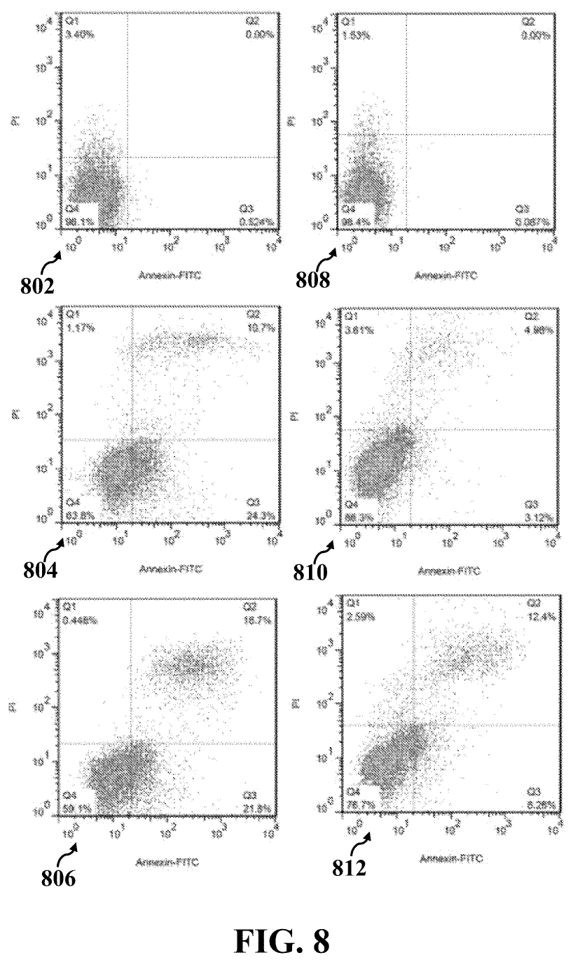

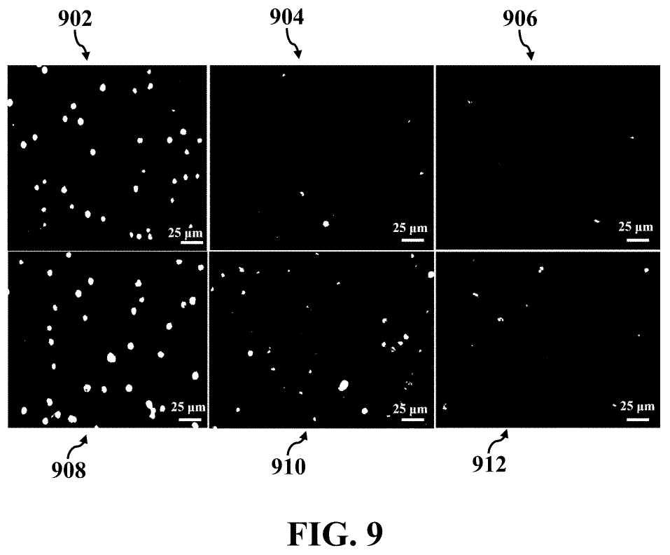

It may be observed from FIG. 6 that the extracted CV signals from the acidified normal cells (MCF10A) exhibited an increase in the oxidation/reduction peak and in the electrical current compared to that of untreated control cells (diagrams 602, 604 and 606). The non-metastatic breast cancer cells, MCF7 cells, behave similar to the normal breast cells (MCF10A) (diagrams 608, 610 and 612), but with a lower slope. However, the CV profile of MDA-MB468 metastatic cells at pH 6.5 (diagram 616) displayed a similar behavior to that of the control (diagram 614), whereas the presence of an oxidation/reduction peak at pH 5.5 (diagram 618) could not be neglected. The reason that there is no anodic/cathodic peak in all of the untreated control cells (diagrams 602, 608, and 614) may be that the compact monolayer of the attached cells on the working electrode does not allow the current flow to happen and as a result, a current blocking occurs. Thus, the increment of the electrochemical response peak observed in the MCF10A and MCF7 in the acidified media may imply that the cells undergone apoptosis and were detached from the working electrode; as a result, the working electrode may detect the whole or a part of the RPMI culture media and the CV profile gets similar to that of RPMI solution (diagrams 604-606 and 610-612). The intensity of the oxidation/reduction peaks may be correlated to the percentage of apoptosis and maintenance of cell viability as confirmed by biological assays shown in FIGS. 8 and 9 in EXAMPLE 3 herein below. Therefore, the lack of electrochemical peaks in the metastatic MDA-MB468 cells in pH 6.5 and pH 5.5 (T0-T8) may be equal to their resistance to apoptosis (diagram 616, diagram 810 of FIG. 8, and image 910 of FIG. 9), yet longer treatments in pH 5.5 would lead to peak emergence and apoptosis initiation (diagram 618).

DPV Responses Monitoring and Analysis:

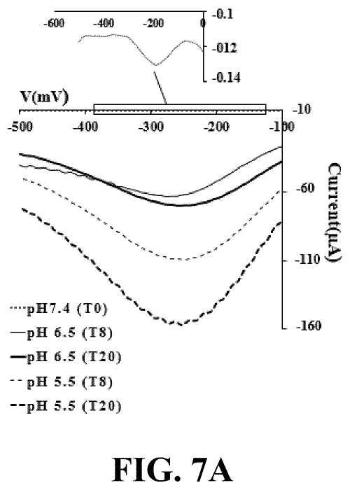

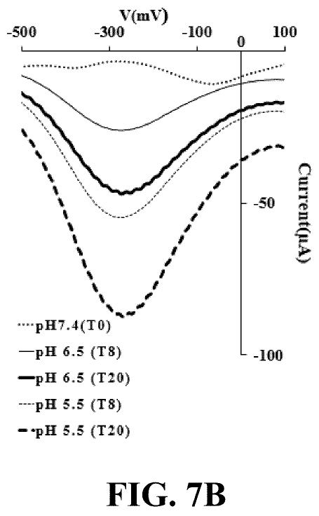

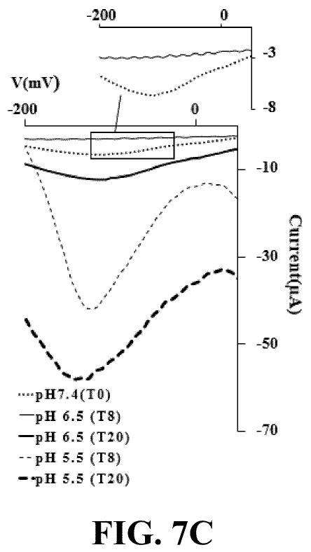

FIGS. 7A-7C show DPV responses of MCF10A cells (FIG. 7A), MCF7 cells (FIG. 7B), and MDA-MB468 cells (FIG. 7C) cultured on nano-roughened working electrode and incubated in different pHs of 7.4, 6.5 and 5.5 for various time intervals of T0 (t=0), T8 (t=8 hours), and T20 (t=20 hours), consistent with one or more exemplary embodiments of the present disclosure. It may be observed that the intensity of the peaks is quite sharper in normal cells and become lower in the progressive cancer cells. Consistent with CV responses data, DPV findings implied that acidosis state may be activated in normal breast (MCF10) cells in the acidic media leading to an enhanced electrochemical peak (FIG. 7A), while the intensity of the peaks was diminished in breast cancer cells regarding their invasive stage (FIGS. 7B and 7C). DPV profile for metastatic MDA-MB468 cells showed a similar behavior in both normal and lower acidic ambient (FIG. 7C).

### Example 3: Biological Assays

To confirm that apoptosis was induced in cells during acidifying process, a complete set of biological assays including Annexin/PI, mitochondrial membrane potential (MMP), Nitrite (NO₂⁻) and intracellular reactive oxygen species (ROS) were carried out. These assays and their results are summarized below.

Annexin/PI (ANPI) Analysis:

Apoptosis percentage was detected by Annexin V-FITC Apoptosis Detection Kit. After culturing MCF10A, MCF7 and MDA-MB468 cell lines in different pH media (pH 6.5, 5.5), cells were trypsinized and collected by centrifugation and re-suspended in about 500 μL of a binding buffer solution. In the next step, about 5 μL of Annexin V-FITC and about 5 μL of Propidium Iodide (PI 50 μg/ml) were added to the cells and after incubation at room temperature for about 5 minutes dark, the fluorescent intensity was measured by flowcytometery.

FIG. 8 shows ANPI results for MCF7 cells at pH values of 7.4 (diagram 802), 6.5 (diagram 804), and 5.5 (diagram 806) (left-side images), and MDA-MB468 cells at pH values of 7.4 (diagram 808), 6.5 (diagram 810), and 5.5 (diagram 812) (right-side images), consistent with one or more exemplary embodiments of the present disclosure. Annexin-PI assay results show the apoptotic states of the cells in 4 states (viable, early apoptose, late apoptose and necrosis). It may be observed that in pH 7.4 (diagrams 802 and 808), that may be an optimum pH level for cell's life, the majority of the cells was in the viable states as normally. MCF7 cells in the low acidic media (pH=6.5) and high acidic media (pH=5.5) entered to early and late apoptosis, respectively (diagrams 804 and 806). Invasive breast cancer cells (MDA-MB468) in the low acidic media (pH=6.5) in contrast of high acidic media (pH=5.5) resisted entering in apoptotic states and kept their viable features (diagrams 810 and 812). So, a meaningful reduction in the fraction of live MCF7 cells may be observed after maintaining in pH=6.5 and pH=5.5. But, the behavior of MDA-MB468 cells in the same acidic culturing media was different in low acidic media from MCF7 cells. ANPI results revealed that MDA-MB468 cells survived in lower acidic media (pH=6.5).

Measurement of the Mitochondrial Membrane Potential (MMP):

Additionally, the effect of acidic extracellular media on MMP, as a main mitochondrial factor that controls the cellular processes, was measured. MMP was assessed by applying rhodamine 123 as a fluorescent dye (Ex/Em=485/535 nm). After treatment, the cells were trypsinized and suspended in about 1 ml Phosphate-buffered saline (PBS). Next, about 3 ml of 1 mg/ml rhodamine 123 was added to cell suspension and incubated for about 10 minutes at 25° C. The cells were then washed twice with PBS and were observed with a fluorescence microscope at 200 magnifications. Also, MMP was measured using a flow cytometer. In brief, about 1×10⁶ cells in a 60 mm culture dish were incubated and then were washed twice with PBS and incubated with rhodamine 123 (0.1 mg/ml) at 37° C. for about 30 minutes. The absence of rhodamine 123 from cells showed the loss of MMP in the cells.

FIG. 9 shows MMP results for MCF7 cells incubated at pH values of 7.4 (image 902), 6.5 (image 904), and 5.5 (image 906) (top images, respectively), and MDA-MB468 cells incubated at pH values of 7.4 (image 908), 6.5 (image 910), and 5.5 (image 912) (bottom images, respectively), consistent with one or more exemplary embodiments of the present disclosure. In fact, MMP reduction would lead to the initiation of apoptosis and the absence of Rhoda mine123 from the cells indicates the loss of MMP. It may be observable that incubating the cells in acidic media decreased the level of MMP in both MCF7 and MDA-MB468, but such reduction was more noticeable in MCF7 cells (images 904 and 906 vs. images 910 and 912).

Nitrite (NO₂⁻) Detection:

For evaluation of the amount of NO release, Griess reagent was utilized under acidic conditions to record the accumulated nitrite (NO₂⁻), which may be a stable breakdown product of NO. During the assay, medium aliquots were mixed with equal volumes of Griess reagent and incubated at room temperature for about 15 minutes. To analyze the azo dye production, a spectrophotometer with absorbance set at 490 nm was used. Sodium nitrite was used as a standard. Griess reagent was applied to investigate the effect of pH treatment on NO₂⁻ production in the metastatic and non-metastatic breast cancer cell lines.

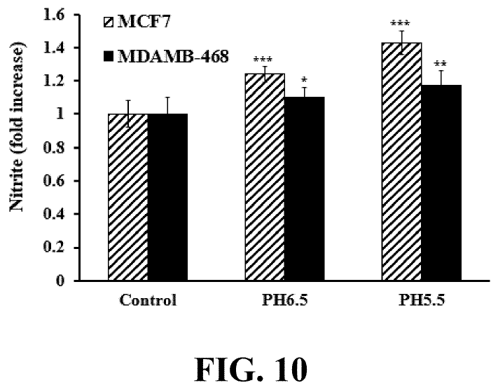

FIG. 10 shows nitrite ion (NO₂⁻) release for MCF7 and MDAMB468 cell lines at three different pH values of 7.4 (Control), 6.5, and 5.5, consistent with one or more exemplary embodiments of the present disclosure. The observation revealed that over production of NO₂⁻ was more significant in MCF7 cells (1.3±0.2, fold compared to the control). In confirmation with previous analyses results mentioned above, at pH 6.5, no major NO₂⁻ over production was observed in MDA-MB468 cell line.

Detection of Intracellular Reactive Oxygen Species (ROS) Levels:

Intracellular ROS were spotted using an oxidation-sensitive fluorescent probe dye, 2, 7-dichlorodihydro fluorescein diacetate (DCFD). Concisely, about 1×10⁶ cells were incubated in a 60 mm culture dish. The cells were then washed in PBS and incubated for about 30 minutes at 37° C. with PBS containing about 20 μM DCFD. The samples were analyzed using a flow cytometer.

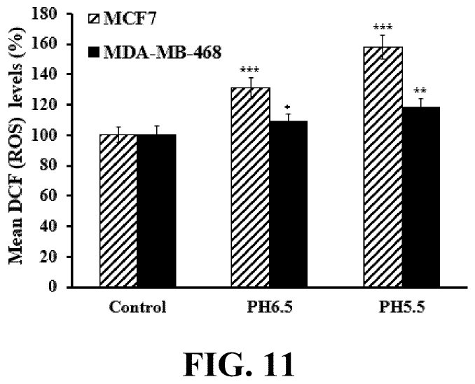

FIG. 11 shows ROS release for MCF7 and MDAMB468 cell lines at three different pH values of 7.4 (Control), 6.5, and 5.5, consistent with one or more exemplary embodiments of the present disclosure. Similar to NO₂⁻ assay, ROS analysis demonstrated that the treatment of MCF7 cells in both acidic pHs increased the intracellular ROS levels. Also, the increment in the level of ROS was milder in acidified MDA-MB468 cells.

Hence, acidic treatment of unknown cells followed by an exemplary monitoring procedure of electrochemical responses of the cells, as disclosed herein, may be utilized to accurately detect type/status of the cells within one group of healthy cells, non-metastatic cancer cells, and metastatic cancer cells. Also, disclosed method and electrochemical biosensor may be utilized for metastasis diagnosis.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter. While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1- A method for detecting status of biological cells, comprising:
culturing a plurality of biological cells on a working electrode of an electrochemical biosensor;
changing extracellular acidity of the plurality of cultured biological cells by adding an acidic solution onto the working electrode;
monitoring an electrochemical response of the plurality of cultured biological cells by monitoring at least one of a cyclic voltammetry (CV) diagram from the plurality of cultured biological cells, a differential pulse voltammetry (DPV) diagram from the plurality of cultured biological cells, and combinations thereof; and
detecting a status of the plurality of cultured biological cells within one of three status groups comprising healthy cells, non-metastatic cancer cells, and metastatic cancer cells based on the monitored electrochemical response.

2- The method of claim 1, wherein:
monitoring the electrochemical response of the plurality of cultured biological cells comprises monitoring a CV diagram from the electrochemical biosensor at least 4 hours after changing the extracellular acidity of the plurality of cultured biological cells, and
detecting the status of the plurality of cultured biological cells comprises detecting the plurality of cultured biological cells within a group of metastatic cells responsive to lack of an oxidation/reduction peak in the CV diagram.

3- The method of claim 1, wherein:
monitoring the electrochemical response of the plurality of cultured biological cells comprises monitoring a set of time-lapsed CV diagrams after changing the extracellular acidity of the plurality of cultured biological cells at time intervals of at least 2 hours, and
detecting the status of the plurality of cultured biological cells comprises detecting the plurality of cultured biological cells within a group of metastatic cells responsive to increasing a peak current of an oxidation/reduction peak of the CV diagrams in the set of time-lapsed CV diagrams with an increasing rate of less than 3 μA/hr.

4- The method of claim 1, wherein:
monitoring the electrochemical response of the plurality of cultured biological cells comprises monitoring a CV diagram from the electrochemical biosensor at least 4 hours after changing the extracellular acidity of the plurality of cultured biological cells, and
detecting the status of the plurality of cultured biological cells comprises detecting the plurality of cultured biological cells within at least one group of healthy cells or non-metastatic cancer cells responsive to observing an oxidation/reduction peak in the CV diagram.

5- The method of claim 4, wherein:
monitoring the electrochemical response of the plurality of cultured biological cells comprises monitoring a set of time-lapsed CV diagrams after changing the extracellular acidity of the plurality of cultured biological cells at time intervals of at least 2 hours, and
detecting the status of the plurality of cultured biological cells comprises detecting the plurality of cultured biological cells within the group of healthy cells responsive to an increasing peak current of a set of oxidation/reduction peaks corresponding to the set of time-lapsed CV diagrams with an increasing rate of 7 μA/hr or more.

6- The method of claim 4, wherein:
monitoring the electrochemical response of the plurality of cultured biological cells comprises monitoring a set of time-lapsed CV diagrams after changing the extracellular acidity of the plurality of cultured biological cells at time intervals of at least 2 hours, and
detecting the status of the plurality of cultured biological cells comprises detecting the plurality of cultured biological cells within the group of non-metastatic cancer cells responsive to an increasing peak current of a set of oxidation/reduction peaks corresponding to the set of time-lapsed CV diagrams with an increasing rate between 3 μA/hr and 7 μA/hr.

7- The method of claim 1, wherein:
monitoring the electrochemical response of the plurality of cultured biological cells comprises monitoring a set of time-lapsed DPV diagrams after changing the extracellular acidity of the plurality of cultured biological cells at time intervals of at least 2 hours, and
detecting the status of the plurality of cultured biological cells comprises one of:
detecting the plurality of cultured biological cells within a group of healthy cells responsive to an increasing rate of more than 100% in a set of peak currents of the set of time-lapsed DPV diagrams;
detecting the plurality of cultured biological cells within a group of non-metastatic cancer cells responsive to an increasing rate between 50% and 100% in the set of the peak currents of the set of time-lapsed DPV diagrams; and
detecting the plurality of cultured biological cells within a group of metastatic cells responsive to an increasing rate of less than 50% in the set of the peak currents of the set of time-lapsed DPV diagrams.

8- The method of claim 1, further comprising fabricating the electrochemical biosensor, comprising:
growing a silicon dioxide (SiO₂) layer on a silicon substrate;
depositing a photoresist layer on the SiO₂ layer;
removing the photoresist layer from the SiO₂ layer inside an area associated with the working electrode by patterning the photoresist layer;
forming a nano-roughened surface on the SiO₂ layer inside the area associated with the working electrode;
removing the photoresist layer from top of the SiO₂ layer;
depositing a gold/titanium (Au/Ti) bilayer on the SiO₂ layer, comprising:
depositing a Ti layer on the SiO₂ layer using radio frequency (RF) sputtering system; and
depositing an Au layer on the Ti layer using the Radio Frequency (RF) sputtering system; and
forming a reference electrode, a counter electrode, and the working electrode by patterning the Au/Ti bilayer using photolithography technique.

9- The method of claim 8, wherein forming the nano-roughened surface on the SiO₂ layer inside the area associated with the working electrode comprises forming nano-features with diameter of less than 100 nm and height of between 100 nm and 150 nm on the SiO₂ layer inside the area associated with the working electrode.

10- The method of claim 8, wherein forming the nano-roughened surface on the SiO₂ layer inside the area associated with the working electrode comprises roughening surface of the area associated with the working electrode by deep reactive ion etching (DRIE) process, comprising:
iteratively etching the surface of the area associated with the working electrode and passivating the surface of the area associated with the working electrode.

11- The method of claim 1, wherein culturing the plurality of biological cells on the working electrode of the electrochemical biosensor comprises adhering the plurality of biological cells onto the plurality of nano-features of the nano-roughened surface of the working electrode.

12- The method of claim 1, wherein culturing the plurality of biological cells on the working electrode of the electrochemical biosensor comprises:
seeding the plurality of biological cells on the working electrode by adding a cell suspension onto the working electrode, the cell suspension comprising a cell line in a cell culture medium with normal pH; and
adhering the cell line to the working electrode by maintaining the electrochemical biosensor with the seeded plurality of biological cells in an incubator.

13- The method of claim 12, wherein culturing the plurality of biological cells on the working electrode of the electrochemical biosensor further comprises producing cell lines of a biological tissue by isolating a plurality of cell lines from the biological tissue.

14- The method of claim 12, wherein maintaining the electrochemical biosensor in the incubator comprises maintaining the electrochemical biosensor with the cell suspension added onto the working electrode in a CO₂ incubator for a time interval between 2 hours and 5 hours.

15- The method of claim 1, wherein changing the extracellular acidity of the plurality of cultured biological cells comprises increasing the extracellular acidity of the plurality of cultured biological cells to a pH value in a range between 5.4 and 6.7.

16- The method of claim 1, wherein monitoring the electrochemical response of the plurality of cultured biological cells, comprising:
applying a direct current (DC) electrical voltage to the working electrode in a range between −0.8 V and 0.8 V; and
extracting the electrochemical response from the electrochemical biosensor.

17- A method for metastasis diagnosis, comprising:
fabricating an electrochemical biosensor comprising a working electrode with a nano-roughened surface, comprising:
forming a nano-roughened surface on an area associated with the working electrode on a silicon dioxide (SiO₂) layer by deep reactive ion etching (DRIE) process;
depositing a gold/titanium (Au/Ti) bilayer on the SiO₂ layer; and
patterning a reference electrode, a counter electrode and the working electrode on the Au/Ti bilayer using photolithography technique;
forming a plurality of cultured biological cells on the working electrode by adhering a plurality of biological cells onto the nano-roughened surface of the working electrode;
changing extracellular acidity of the plurality of cultured biological cells to a pH value of the extracellular environment of the plurality of cultured biological cells in a range between 5.4 and 6.7 by adding an acidic solution onto the working electrode;
applying an electrical voltage to the working electrode in a range between −0.8 V and 0.8 V;
extracting a cyclic voltammetry (CV) electrochemical response from the electrochemical biosensor; and
detecting a presence of metastatic cells in the plurality of cultured biological cells responsive to a lack of an oxidation/reduction peak in the CV electrochemical response.
